(19) United States
(12) Patent Application Publication
(10) Pub. No.: US 2024/0206723 A1
(43) Pub. Date: Jun. 27, 2024

(54) SYSTEMS FOR SELF-REFRACTION AND REMOTE EYE EXAMS OF HUMAN EYES

(71) Applicants: Junzhong Liang, Fremont, CA (US); Ling Yu, Fremont, CA (US)

(72) Inventors: Junzhong Liang, Fremont, CA (US); Ling Yu, Fremont, CA (US)

(21) Appl. No.: 18/557,113

(22) PCT Filed: Apr. 27, 2022
(86) PCT No.: PCT/US22/26458

§ 371 Date: Oct. 25, 2023

Related U.S. Application Data
(60) Provisional application No. 63/258,467, filed on May 3, 2021.
(60) Provisional application No. 63/258,468, filed on May 3, 2021.

(51) Int. Cl. A61B 3/028; A61B 3/00; A61B 3/032

(52) U.S. Cl. CPC A61B 3/0285; A61B 3/0033; A61B 3/032

(57) ABSTRACT

A refraction system for remote refraction or self-refraction of human eyes. The system uses a reliable spherocylindrical module that allows the refraction system to obtain an initial spherocylindrical correction of the eye, and an SPH adjustment module that allows the user to adjust spherical power of the phoropter module on top of the initial spherocylindrical correction of the eye so that an updated spherical power (SPH) is subjectively determined. The reliable spherocylindrical module can be I) a device for obtaining a prescription of a pair of old eyeglasses, or II) a wavefront aberrometer that can offer, in addition to the objective sphero-cylinder correction, a quality metrics for at least one of a) measuring the confidence level in the objectively determined cylinder power and cylinder axis, b) assessing/displaying quality of vision corrections for a plurality of cylinder power.

### RELATED APPLICATIONS

This application claims priority to the U.S. Provisional Patent Application No. 63/258,467, filed on May 3, 2021 by Junzhong Liang and Ling YU, entitled “Systems for Self-Refraction and Remote Eye Exams of Human Eyes,” and it is ALSO related to U.S. Provisional Patent Application No. 63/258,468, filed on May 3, 2021 by Junzhong Liang and Ling YU, entitled “Methods and Systems for Optimizing Refractive Refraction of Human Eyes”; all of which are hereby incorporated by reference in full.

### BACKGROUND

The invention relates generally to systems and methods for eye exams and refraction of human eyes.

Conventional refraction processes rely on the experience and skills of an individual eye care professional (e.g., an optometrist or optician) to set the starting and ending points of a spherical power, a cylinder power, and a cylinder axis for an eyeglass prescription.

A block diagram 1 representing a conventional refraction process is shown in FIG. 1. First, an autorefractor 11 is typically used to take an objective measurement of an eye's refractive errors and provide a rough objective prescription in objective refraction step 12, where the objective prescription includes an objective spherical power Fₛ, an objective cylinder power F_{c} and an objective cylinder angle Fₐ. Second, an eye care professional determines a rough spherical correction in a phoropter 13, and then administrates a subjective optimization of spherical power, cylinder power and cylinder angle based on the objective prescription from step 12. The subjective optimization is based on the experience and skill of the optometrist or optician, and on subjective feedback of the tested subject (i.e., the patient).

Steps 16, 17 and 18 are part of the subjective refraction performed using the phoropter 13. In step 16, the cylinder angle Fₐ is subjectively optimized by letting the tested subject first see an astigmatism chart and then an acuity chart afterwards. The eye care professional will set and modify the cylinder angle by an amount δFₐ based on the objective prescription of step 12 as well as feedback of the tested subject. In step 17, the cylinder power F_{c} is subjectively optimized by having the tested subject view an acuity chart, and an eye care professional will set and modify the cylinder power by an amount δF_{c} based on the objective prescription as well as feedback of the tested subject. In step 18, the spherical power is subjectively optimized by letting the tested subject see an acuity chart, and an eye care professional will set and modify the spherical power Fₛ by an amount δFₛ based on feedback of the tested subject. The same process of steps 16, 17 and 18 are repeated for the other eye of the tested subject. In subjective refraction step 14, a final prescription of the eyeglasses is determined for each eye using the subjectively optimized spherical power Fₛ+δFₛ of step 18, the subjectively optimized cylinder power F_{c}+δF_{c} of step 17, and the subjectively optimized cylinder angle Fₐ+δFₐ of step 16.

The conventional refraction process as shown in FIG. 1 relies on experience and skills of individual optometrist (optician) to set the starting as well as the ending points of a spherical power (SPH), a cylinder power (CYL), and a cylinder axis (AXIS) for the eyeglasses. This conventional approach is subjective and has at least three drawbacks. First, the process, relying on experience of optometrist (optician), cannot be standardized because each optometrist (optician) has his/her own experience in the past, which will change over time for each optometrist (optician). Second, if patients do not like the new eyeglasses, optometrists (opticians) make personal adjustments to the new prescription and the new eyeglasses are made for the patients. Remaking new eyeglasses is common today, and may take a few iterations until patients finally accept a new pair of eyeglasses. Third, a pair of new eyeglasses may take 1 to 2 weeks for consumers to get used to, and individual's experience varies from person to person. It could be painful process for some consumers sometimes. A significant portion of new eyeglasses purchased are abandoned because consumers can never get used to them.

Refraction of human eyes or eye exams are only performed in individual optometry offices and ophthalmology offices in the US, or optical shops in some countries. Disadvantages include prescriptions of eye glasses are expensive about 40$ to $75 in the US, and eyeglasses are expensive in optical shops.

Although self-refraction using software APPs is available, it is however limited and unreliable. Outdated prescriptions can be verified by measuring subjective visual acuity using software APPs. If visual acuity remains to be 20/20 or better with the existing eyeglasses, the old prescription can be declared still valid and re-prescribed by a certified optometrist or an ophthalmologist. However, if visual acuity is degraded with the old eyeglasses or refractive properties of eyes have changed from the last prescription, self-refraction using software APPs will be impossible or no longer reliable.

Consequently, although configurations and methods for self-refraction are known in the art, all of them suffer from one or more disadvantages. Thus, there is a need to provide systems and methods of self-refraction or remote eye exams.

### SUMMARY

The present invention provides a refraction system for remote refraction or self-refraction of human eyes, comprising: a) a phoropter module that allows to place a plurality of optical lenses in front of a tested eye for refractive corrections; b) a vision chart module that displays letters or pictures for the tested eye to observe and to determine the best corrected visual acuity BCVA by a tested subject; c) a computer module that provides control to the refraction system; d) a communication module that allows a user or an operator to communicate with the computer module, wherein the user is the subject under test and an operator is someone who assists the user for a refraction test, and communicating with the computer module includes recording the best corrected visual acuity for the eye; e) a reliable spherocylindrical module that allows the refraction system to obtain an initial spherocylindrical correction of the eye, wherein the spherocylindrical error consists of a spherical power (SPH1), and a cylinder power (CYL) and a cylinder (AXIS), wherein the cylinder power (CYL) and a cylinder (AXIS) in known to be accurate and reliable; f) an SPH adjustment module that allows the user to adjust spherical power of the phoropter module on top of the initial spherocylindrical correction of the eye so that an updated spherical power (SPH) is subjectively determined; g) an output module that allows to present a new refractive prescription that includes the updated spherical power SPH, the cylinder power CYL and cylinder AXIS, and the best corrected visual acuity BCVA.

### BRIEF DESCRIPTION OF THE DRAWINGS

FIG. 1 shows a block diagram of a conventional refraction process.

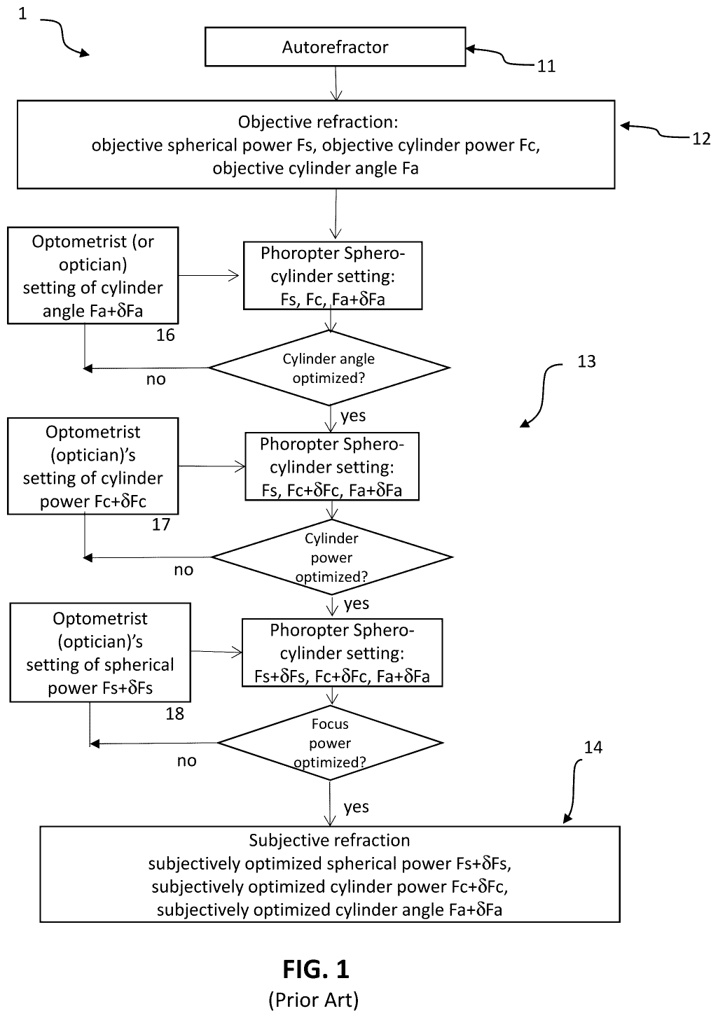

FIG. 2 shows a schematic diagram of a system for self-refraction and remote-refraction in accordance with some embodiments.

FIG. 3 shows a schematic diagram of Kiosk for self-refraction and remote refraction in accordance with some embodiments.

### DETAILED DESCRIPTION

Reference now will be made in detail to embodiments of the disclosed invention, one or more examples of which are illustrated in the accompanying drawings. Each example is provided by way of explanation of the present technology, not as a limitation of the present technology. In fact, it will be apparent to those skilled in the art that modifications and variations can be made in the present technology without departing from the spirit and scope thereof. For instance, features illustrated or described as part of one embodiment may be used with another embodiment to yield a still further embodiment. Thus, it is intended that the present subject matter covers such modifications and variations as come within the scope of the appended claims and their equivalents.

In order to address the issues of expensive eye exams as well as expensive eyeglasses, we disclose a new system for self-refraction by the tested subject and remote refraction that can be operated by technicians with supervision by certified optometrists and ophthalmologists at centralized remote locations.

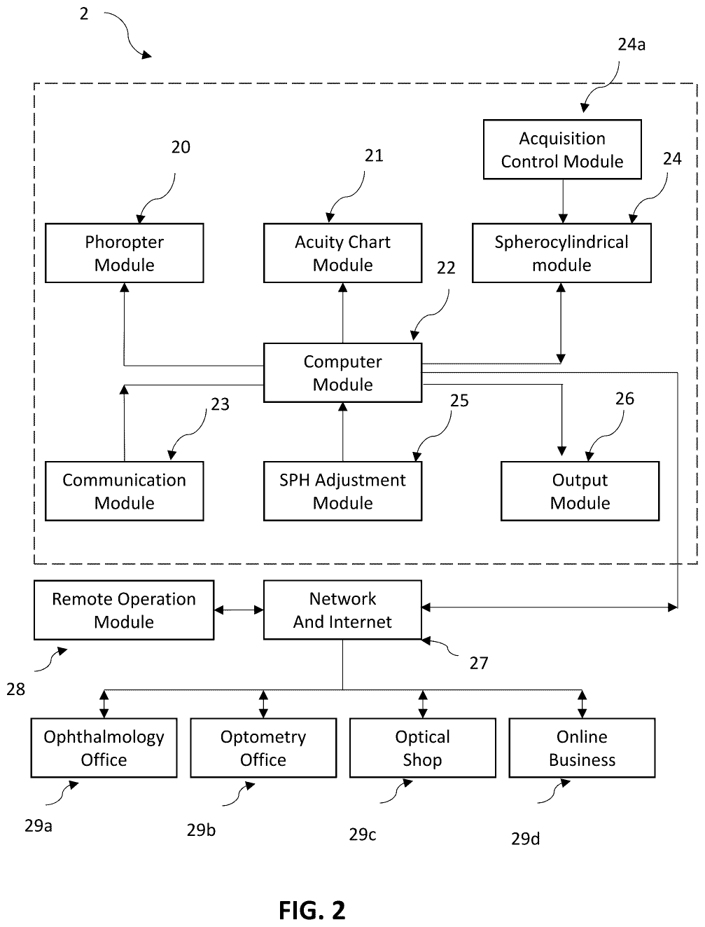

In one aspect of the present invention, the refraction system 2 in FIG. 2 comprise: a) a phoropter module 20 that allows to place a plurality of optical lenses in front of a tested eye for refractive corrections; b) a vision chart module 21 that displays letters or pictures for the tested eye to see so that the best corrected visual acuity BCVA is determined by a tested subject; c) a computer module 22 that provides control to the refraction system; d) a communication module 23 that allows a user or an operator to communicate with the computer module, wherein the user is a subject under test and an operator is someone who assists the user (tested subject) for a refraction test, and communicating with the computer module includes recording the best corrected visual acuity for the eye (BCVA); e) a reliable spherocylindrical module 24 that allows the refraction system to obtain an initial spherocylindrical correction of the eye, wherein the spherocylindrical error consists of a spherical power (SPH1), and a cylinder power (CYL) and a cylinder (AXIS) while the cylinder power (CYL) and a cylinder (AXIS) is known to be accurate and reliable; f) an SPH adjustment module 25 that allows the user (tested subject) to adjust spherical power of the phoropter module so that an updated spherical power (SPH) is subjectively determined; g) an output module 26 that allows to present a new refractive prescription that includes the updated spherical power SPH, the cylinder power CYL and cylinder AXIS, and the best corrected visual acuity BCVA.

Comparing to self-refraction using software APPs, the system in FIG. 2 has at least four advantages.

Firstly, it provides a mechanism to input an prescription of an pair of eyeglasses, and more importantly another mechanism to modify the prescription in order to obtained the best corrected visual acuity for the tested eye. In one embodiment, inputting an prescription of an pair of eyeglasses can be achieved using the reliable spherocylindrical module 24 such as a lensometer module with which a pair of eyeglasses for the tested subjects can be measured. In another embodiment, inputting an prescription of an pair of eyeglasses can be achieved by using the communication module 23 that includes a keyboard. The old prescription for the tested eye can be dialed-into the phoropter module 20, and more importantly the old prescription can be adjusted by the tested subject and using the SPH adjustment module 25. Embodiments of the SPH adjustment module include but are not limited to a) a knob module that can be turned by the user for changing spherical power of the phoropter module; b) a voice-controlled module that can use user's voice to change spherical power of the phoropter module; c) a plurality of buttons that that can be pushed by the user to change spherical power of the phoropter module.

Secondly, since refraction properties of a human eye can change significantly after the last eye exam or the tested subject may not have a pair of eyeglasses and its prescription, the refractions system in the present invention in FIG. 2 further enables to measure the refractive properties for the tested eye using the reliable spherocylindrical module 24. This allows to obtain a completely new prescription that can be different from the prescription of an old eyeglasses not only in SPH power, but also in CYL power and in cylinder AXIS. In one embodiment, the reliable spherocylindrical module is an objective refraction module with which the tested eye can be measured for obtaining an initial spherocylindrical correction of the tested eye. The objective refraction module can be further configured as a wavefront aberrometer which measures not only an initial spherocylindrical correction of the tested eye but also high-order optical aberrations in the eye. Using all the aberrations in the eye, which include the initial spherocylindrical correction and the high order optical aberrations in the eye, the computer module 22 performs a global search of a new and optimized estimation of spherical error (SPH1), cylinder power (CYL) and cylinder orientation (AXIS).

Thirdly, once the optimized cylinder power (CYL) and cylinder AXIS as well as a subjectively optimized spherical power (SPH) is determined for a pair of eyeglasses, optical vision diagnosis can be derived from the calculated retinal images from the residual aberrations in the eye. Thus, comprehensive eye exams can be obtained, which includes subjectively determined best corrected visual acuity (BCVA), optical vision diagnosis based on calculated retinal images, as well as a prescription based on subjectively determined spherical power SPH, objectively optimized cylinder power CYL and cylinder AXIS.

Fourthly, the system in FIG. 2 in one embodiment is further configured to have an acquisition control module 24a for the tested subject to trigger and review wavefront measurements himself or herself.

A plurality of optical lenses in the phoropter module in one embodiment are lenses with fixed refractive powers or lenses with refractive properties that are electrically adjusted.

In one embodiment, the communication module 23 in FIG. 2 includes but is not limited to a keyboard and the communication with the computer module further includes inputting personal information.

In another embodiment, the output module of the refraction system in FIG. 2 includes but is not limited to a) a printer that is connected to the computer module, b) a display device that is connected to the computer module.

In another aspect of the present invention, the refraction system in FIG. 2 is configured for remote refraction so that an operator who is physically away from the refraction system can perform remote refraction of a tested subject.

In one embodiment, the system for remote refraction is configured to be connected to a communication network and internet 27.

In another embodiment, the system for remote refraction is further configure to have a remote operation module 28 so that an operator can control the refraction system remotely.

In yet another embodiment, the system for remote refraction is further configured to have a video/audio communication channel between the operator and the tested subject.

In still another embodiment, the operator includes technicians under supervision by a certified optometrist for providing an official prescription for a pair of eyeglasses.

In one embodiment, the system for remote refraction is further connected to one of the following businesses: a) an ophthalmology office 29a, b) an optometry office 29b, c) an optical shop 29c, d) an online business organization 29d.

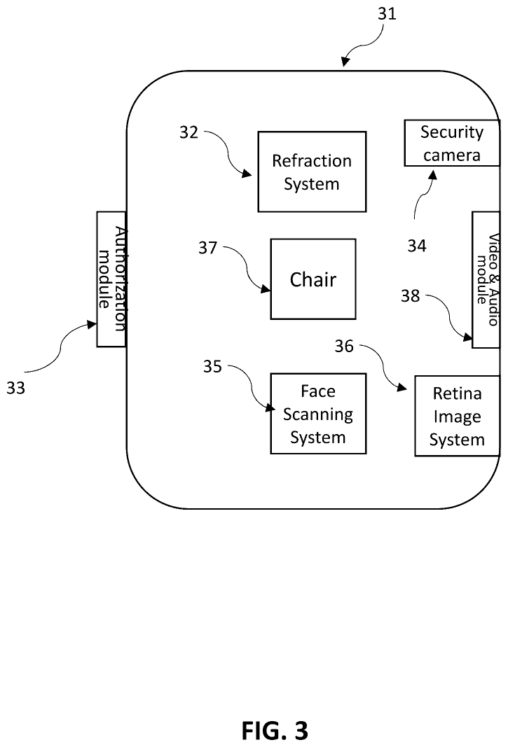

In yet another aspect of the present invention, the refraction system in FIG. 2 is further configured to be part of a kiosk for eye exam in FIG. 3. In one embodiment, the refraction system 32 is placed inside an enclosure 31 along with at least one of the followings: a) an authorization module 33 to secure the Kiosk door so that only authorized customers can use the Kiosk, b) a monitor system 34 that uses at least a security camera to provide a real-time monitor of the Kiosk, c) an image module or a face scanning system 35 for measuring pupil distances of the tested eyes, d) a retinal image system module 36 such as an OCT, a fundus camera, a laser scanning ophthalmoscope, e) a chair module 37 for the tested subject to be properly positioned in the Kiosk, f) a video and audio module 38 for the customer to communicate with technicians and clinicians at a remote location. In one embodiment, the face scanner module 35 acquires images of the tested subject in a plurality of perspectives, and acquired images can be used for trying-on eyeglasses frames online.

Reference has been made in detail to embodiments of the disclosed invention, one or more examples of which have been illustrated in the accompanying figures. Each example has been provided by way of explanation of the present technology, not as a limitation of the present technology. In fact, while the specification has been described in detail with respect to specific embodiments of the invention, it will be appreciated that those skilled in the art, upon attaining an understanding of the foregoing, may readily conceive of alterations to, variations of, and equivalents to these embodiments. For instance, features illustrated or described as part of one embodiment may be used with another embodiment to yield a still further embodiment. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only and is not intended to limit the invention.

What is claimed is:

1. A refraction system for self-refraction or remote refraction of human eyes, comprising:
a) a phoropter module that allows to place a plurality of optical lenses in front of a tested eye for refractive corrections;
b) a vision chart module that displays letters or pictures for the tested eye to see and to determine the best corrected visual acuity BCVA by a tested subject;
c) a computer module that provides control to the refraction system;
d) a communication module that allows a user or an operator to communicate with the computer module, wherein the user is the subject under test and an operator is someone who assists the user (tested subject) for a refraction test, and communicating with the computer module includes recording the best corrected visual acuity for the eye BCVA;
e) a reliable spherocylindrical module that allows the refraction system to obtain an initial spherocylindrical correction of the eye, wherein the spherocylindrical error consists of a spherical power (SPH1), and a cylinder power (CYL) and a cylinder (AXIS), wherein the cylinder power (CYL) and a cylinder (AXIS) in known to be accurate and reliable;
f) a SPH adjustment module that allows the user or an operator to adjust spherical power of the phoropter module on top of the initial spherocylindrical correction of the eye so that an updated spherical power (SPH) is subjectively determined;
g) an output module that allows to present a new refractive prescription that includes the updated spherical power SPH, the cylinder power CYL and cylinder AXIS, and the best corrected visual acuity BCVA.

2. The system of claim 1, wherein a plurality of optical lenses are lenses with fixed refractive powers or lenses with refractive properties that are electrically adjusted.

3. The system of claim 1, wherein the communication module includes but is not limited to a keyboard and the communication with the computer module includes but not is limited to
a) inputting personal information;
b) inputting data from an old prescription;

4. The system of claim 1, wherein the reliable spherocylindrical module is a lensometer module with which a pair of eyeglasses can be measured, and from which an initial spherocylindrical correction of the tested eyes can be communicated to the computer module.

5. The system of claim 1, wherein the reliable spherocylindrical module is an objective refraction module with which the tested eye can be measured for obtaining an initial spherocylindrical correction of the tested eye.

6. The system of claim 5 wherein the objective refraction module is a wavefront aberrometer which measures not only an initial spherocylindrical correction of the tested eye but also all the other optical aberrations in the eye.

7. The system of claim 6 further include providing a range of cylinder power with the objective cylinder power (CYL_o) for at least some eyes or determining a quality metrics for at least one of
a) measuring the confidence level in the objectively determined cylinder power and cylinder axis in addition to the objective sphero-cylinder correction,
b) assessing/displaying quality of vision corrections for a plurality of cylinder power.

8. The system of claim 7 further includes using the quality metrics to perform a subjective refraction with a phoropter in a plurality of modes: I) one mode for the subjective determination of spherical power only, II) one mode for the subjective determination of both sphere power and cylinder power.

9. The system of claim 6 is further configured to provide optical vision diagnosis derived from all the other optical aberrations in the eye.

10. The system of claim 6 is further configured to have an acquisition control module for the tested subject to trigger and review wavefront measurements himself or herself.

11. The system of claim 1, wherein the SPH adjustment module is achieved using one of followings:
a) a knob module that can be turned by the user for changing spherical power of the phoropter module;
b) a voice-controlled module that can use user's voice to change spherical power of the phoropter module;
c) a plurality of buttons that that can be pushed by the user to change spherical power of the phoropter module.

12. The system of claim 1, wherein the output module includes but is not limited to
a) a printer that is connected to the computer module;
b) a display device that is connected to the computer module.

13. The system of claim 1 is further configured to be connected to a communication network and internet for remote refraction.

14. The system of claim 1 is further configured to have a video communication channel between the operator and the tested subject.

15. The system of claim 1 is further configured to have an audio communication channel between the operator and the tested subject.

16. The system of claim 13 through 15, the operator includes technicians that is supervised by a certified optometrist for providing an official prescription for a pair of eyeglasses.

17. The system of claim 13 is further connected to one of the following businesses:
a) an optical shop;
b) an ophthalmology office;
c) an optometry office;
d) an online business organization.

18. The system of claim 1 is placed in an enclosure and configured as a Kiosk.

19. The system of claim 18 is further configured to have an authorization module to secure a door of the Kiosk.

20. The system of claim 18 is further configured to have a real-time video monitor system using at least a video camera.

21. The system of claim 18 is future configured to have an image module for measuring pupil distances of the tested eyes.

22. The system of claim 21 wherein the image module is further configured as a face scanner module that acquire image of the tested subject in a plurality of perspectives, wherein the acquired images can be used for trying-on eyeglasses frames online.

23. The system of claim 18 is further configured to have a retina image system such as an OCT, a fundus camera, a laser scanning ophthalmoscope so that retinal images of the tested eyes can be acquired in the Kiosk.
